(19) United States
(12) Patent Application Publication
(10) Pub. No.: US 2007/0190125 A1
(43) Pub. Date: Aug. 16, 2007

(54) Positioning feature for aiding use of film or strip product

(76) Inventors: Constantine Georgiades, East Brunswick, NJ (US); Alexander Borodin, Monmouth Junction, NJ (US); Peter Costello, Franklin Lakes, NJ (US); Jacqueline LePage, Boonton, NJ (US); Markus Krumme, Denville, NJ (US); Wolfgang Schaefer, Madison, NJ (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 11/705,263

(22) Filed: Feb. 12, 2007

Related U.S. Application Data
(63) Continuation-in-part of application No. 11/086,517, filed on Mar. 22, 2005.
Application No. 11/086,517 is a continuation-in-part of application No. 11/030,846, filed on Jan. 7, 2005.
Application No. 11/030,846 is a continuation-in-part of application No. 10/792,362, filed on Mar. 3, 2004.

(51) Int. Cl. A61F 13/02; A61L 15/16

(52) U.S. Cl. 424/448

(57) ABSTRACT

A film or strip product is provided with a positioning feature formed to aid a user in differentiating or distinguishing or otherwise identifying one or more features of the film or strip product. The positioning feature thus may be used to facilitate proper or desired use of the film product upon identification of a desired feature thereof. In one embodiment, the positioning feature is configured to aid in properly orienting and/or positioning and/or applying the film or strip product. The film or strip product may provide a therapeutic affect to a desired treatment site, and may require positional orientation before application to the treatment site, and/or positional orientation with respect to the treatment site. Various positioning features that may serve such function include visual or tactile features that either visually or tactilely indicate to a user sufficient information about the film product to permit the user to use and/or apply and/or orient the film product as desired or required.

### CROSS-REFERENCE To RELATED APPLICATION

The present application is a continuation-in-part and claims the benefit of the earlier filing date of co-pending U.S. patent application Ser. No. 11/086,517, filed Mar. 22, 2005, which application is a continuation-in-part and claims the benefit of the earlier filing date of U.S. patent application Ser. No. 11/030,846, filed Jan. 7, 2005, which application is a continuation-in-part of and claims the benefit of the earlier filing date of U.S. patent application Ser. No. 10/792,362, filed Mar. 3, 2004, all of which applications are hereby incorporated by reference herein in their entireties.

### FIELD OF THE INVENTION

The present invention relates to a positioning feature on a film or strip type product, the positioning feature being provided to differentiate or distinguish features of the film or strip type product. More particularly, the present invention relates to a positioning feature that assists a user in orienting and/or positioning and/or applying a film or strip type product configured to provide a therapeutic effect at a selected body area. Even more particularly, the film or strip type product of the present invention may carry an active for delivery to the desired body area for treatment of such body area.

### BACKGROUND OF THE INVENTION

A variety of topically-applied products, including strips, films, patches and the like, are known in the art. Such products are particularly useful where a protectant film is recommended or where drug or medication retention is desirable.

Film protectants are particularly desirable in situations where wounds or surface openings are present and must be protected. Alternatively, where a drug or medication is easily removed by rinsing or wiping the application area (e.g., transdermal applications), mechanical retention of the drug or medication becomes particularly desirable.

Most recently, strip or film type products have enjoyed renewed popularity in the oral care field. Particular interest has been paid to the areas of teeth whitening and oral transdermal delivery of drugs and medications.

Although a variety of strip or film type products have been disclosed, there still remains a need for improved film or film-like compositions which reduce the inconvenience or discomfort typically associated with the attachment of such foreign objects to sensitive parts of the body. There also remains a need for improved film or film-like compositions which are easier to use and to apply.

For instance, one disadvantage observed regarding the aforementioned film or strip products relates to the eventual need to peel off or in some other way to remove and to discard the film or strip product after delivery of the topical or systemic active.

A related disadvantage is that typically an adhesive material is required to maintain the film or strip product on the desired area until treatment is complete. Such adhesive material may leave a residue at the treatment site. Moreover, when an adhesive material is provided, generally a releasable backing strip is also provided to protect the adhesive material from accumulating debris and also to prevent the film or strip product from adhering to the wrong object or to itself. Such releasable backing strip must be peeled off so that the film or strip product may be adhered to the desired body area for delivery of the active, thereby adding additional inconveniences, such as the need to remove and discard an element, and the need to prevent adherence of the film or strip product to itself.

The inventors of parent U.S. patent applications Ser. Nos. 11/086,517, 11/030,846, and 10/792,362 (the “parent applications,” which are incorporated by reference herein in their entireties) have discovered that film compositions comprising select water insoluble polymers and a disintegration facilitator selected from the group consisting of a plasticizer, a water insoluble particulate, or mixtures thereof is one way to address issues around “peeling” since the above ingredients provide film compositions having good protective properties as well as improved disintegration properties. Such film compositions may be formed into a film product that facilitates use of the product and application thereof to a desired body area to deliver an active, such as a systemic or topical active, to the desired body area.

The film products disclosed in the parent applications may have protective properties such that the film prevents foreign substances, chemicals, or actives from crossing from one side the film to the other. Additionally, or alternatively, the disclosed film products may have controlled (or an extended type or prolonged) disintegration or dissolution properties in aqueous environments. For instance, the film product may deliver the topical or systemic active and thereafter dissolve within a predetermined amount of time.

However, it is possible to form such film products such that a dry adhesive layer is provided such that no releasable backing strip is required. As such, the surface bearing the active may not be readily distinguishable from the opposite surface which may not comprise and/or deliver an active. In such case, it would be desirable to differentiate the active-bearing surface from the inactive surface without affecting the ease with which the film product may be applied or used. It will be appreciated that other types of film products with functionally different surfaces that cannot readily be distinguished from each other are available with similar inconveniences associated with use and application of the correct surface.

### SUMMARY OF THE INVENTION

In accordance with the principles of the present invention, a positioning feature is provided on a film or strip product (hereinafter, simply “film product(s)” for the sake of simplicity and without intent to limit such term) to aid in differentiating or distinguishing features of the film product. Differentiation of features of a film product is helpful for various purposes, including, without limitation, facilitating orientation of the film product. A particular orientation of the film product may be necessary such as for proper application and/or delivery of an active carried by the film product to a desired body area.

According to one aspect of the invention, the film product may be formed such that only one surface thereof is configured to deliver or to apply an active or such that one side delivers one active and the opposite side delivers a second active optionally different in identity and function from the first active. As such, the positioning feature is provided to indicate to a user the proper or appropriate surface to apply to the treatment site so that the active may be delivered to such site.

The positioning feature may be in any desired form capable of achieving its purpose of distinguishing features of the film product, such as for purposes of orienting the film product. For instance, an irregularity in shape or texture or visual feature may be provided along the film product. If the film product is symmetrical about a midline, then the positioning feature may be provided at a position offset from the midline to distinguish the surfaces of the film product from each other.

These and other features and advantages of the present invention will be readily apparent from the following detailed description of the invention, the scope of the invention being set out in the appended claims.

### BRIEF DESCRIPTION OF THE DRAWINGS

The detailed description will be better understood in conjunction with the accompanying drawings, wherein like reference characters represent like elements, as follows:

FIG. 1 is a plan view of an exemplary film product having an exemplary positioning feature in accordance with the principles of the present invention;

FIG. 2 is a plan view of a film product similar to that of FIG. 1, but with a different exemplary positioning feature;

FIG. 3 is a plan view of a film product similar to that of FIG. 1, but with a different exemplary positioning feature;

FIG. 4 is a plan view of a film product similar to that of FIG. 1, but with a different exemplary positioning feature;

FIG. 5 is a plan view of another exemplary film product having an exemplary positioning feature similar to that of FIG. 3; and

FIG. 6 is a perspective view of yet another exemplary film product, with a another type of exemplary positioning feature.

### DETAILED DESCRIPTION OF THE INVENTION

In accordance with the principles of the present invention, an indicator or positioning feature (hereinafter “positioning feature” for the sake of convenience, without intent to limit such term) is provided on a product that is to be applied to a desired treatment site or body area (hereinafter “treatment site” for the sake of convenience, without intent to limit such term). Positioning features formed in accordance with the principles of the present invention are particularly configured to aid the user in differentiating or distinguishing features of the film product. A further purpose of such positioning features may be to aid the user in orienting the product so that the product may perform its desired treatment function effectively. Such positioning features are particularly important, if not necessary, on products that cannot perform their desired functions unless properly positioned or oriented in a particular manner. However, such positioning features are helpful even if not essential to the proper functioning of the product. Products utilizing the positioning features of the present invention are referenced herein as “film products” for the sake of convenience, such term being used in the broad sense to include not only films, but also strips, patches, pads, layers, wafers, and the like, and should not be understood as necessarily limited to film-like properties.

A common function of film products modified by the present invention is to perform a therapeutic function at a selected treatment site in any of a variety of manners. Therapeutic functions include, without limitation, providing a barrier function (such as covering the site to protecting it from contamination or injury, such as by the use of a bandage); or delivering an active, either systemically (such as transcutaneous deliver of a medicament) or topically (such as application of a bleaching agent to teeth, or an antibiotic or analgesic to a dermal wound). The film product may itself have to be positioned in a particular orientation before application to the treatment site, or may have to be applied in a particular orientation with respect to the treatment site.

The film products of the present invention can comprise, consist of, or consist essentially of the essential elements and limitations of the invention of the parent applications, as well as any desirable additional or alternative or optional ingredients, components, or limitations known in the art. Exemplary film products modified by the present invention are disclosed in the parent applications. The disclosed films (either single, bi-, or multi-layered) have barrier as well as controlled disintegration properties. The films disclosed in the parent applications are particularly formed for application to the teeth, oral mucosa, or other affected area of the skin or mouth and allowed to disintegrate over time in the presence of oral fluids or other aqueous media. Examples of oral conditions these substances may address include, but are not limited to, one or more of appearance and structural changes to teeth, whitening, stain bleaching, stain removal, plaque removal, tartar removal, cavity prevention and treatment, inflamed and/or bleeding gums, mucosal wounds, lesions, ulcers, aphthous ulcers, cold sores, tooth abscesses, tooth and/or gum pain, tooth sensitivity (e.g., to temperature changes), and the elimination of mouth malodor resulting from the conditions above and other causes such as microbial proliferation. Additionally, the disclosed films may be useful for treating and/or preventing wounds, lesions, ulcers, cold sores and the like of the lips and skin generally. However, it will be appreciated that a positioning feature formed in accordance with the principles of the present invention may be applied to film products other than simply those disclosed in the parent applications. Moreover, the principles of the present invention need not be limited by a particular condition to be treated. The positioning features of the present invention are useful for effective use of film products in treating any of a variety of conditions, whether oral or dermal or systemic or otherwise.

Film products to be modified by the present invention may be formed to provide any of a desired variety of functions, such as, without limitation, a therapeutic function. A typical therapeutic function is that of affecting a treatment site, such as by application delivery of an active to or at such site. Various topical and systemic actives can be incorporated into film products of the present invention, such as disclosed in the parent applications. The term “topical or system active” as used herein includes, without limitation, curative, prophylactic, and cosmetic active substances or compositions thereof, and includes any actives known in the art. For the sake of brevity, reference is made to the parent applications (which have been incorporated by reference herein) for exemplary oral care actives, as well as other inactives, that may be used in the present invention (including suitable topical actives, and levels thereof, for use in and around the oral cavity).

Topical or system actives that may be provided in film products to be used in the present invention may be deposited upon the surface of the film products. For example, a gel may be deposited directly as a layer on a surface of the film product. Alternatively a gel may be absorbed into the film product, or impregnated into the bulk of the film material, or deposited between layers of a multiple layered film.

Methods of depositing substances upon the surfaces of film materials are known, and include, without limitation, printing (e.g., silo screen printing), passing between impregnated rollers, dosing, a pump and nozzle, spraying, dipping, etc. Methods of impregnating substances into the bulk of film materials are also known, and include, without limitation, admixing the substance into the strip material and then forming the strip, or exposure of the strip to the substance under conditions which cause the substance to be impregnated into the strip. Alternatively, one example of the film material may be a foam material, particularly an open-cell foam material, and the substance may be impregnated into the strip material by introducing the substance into the cells of the foam.

As described in the parent applications, the disclosed film may be marked with one or more visible symbols, e.g., text matter, a trade mark, a company logo, an area of color, or an alignment feature such as a visible line or notch etc. to assist the user in applying the device to the teeth in a proper alignment. Such an alignment feature may, for example, comprise a symbol to show the user which product configuration is “up” whilst applying the product to the teeth, which of a pair of the products is intended for the upper teeth and which for the lower teeth, or which side of a pair of opposite sides comprises the active or a particular active. This way, the product may be made more visually attractive and/or easier to use and/or more effective.

Such symbol(s) may be applied by conventional printing or embossing processes (e.g., silk screen printing, inkjet printing, etc.) to the surface of the plastically deformable material opposite to the surface on which is attached the layer of an absorbent material. If such a visible symbol is applied to this surface, a cover layer can, optionally, be applied over the symbol, for example to protect it. This cover layer may be transparent or translucent to allow visible symbols to be seen through this layer. Such a cover layer can, optionally, be applied to the film by pressing, e.g., rolling, the material of the cover layer in contact with the film.

The present invention elucidates and elaborates upon the provision of an alignment or positioning feature as disclosed in the parent applications. The positioning feature preferably is formed and/or located to aid the user in differentiating or distinguishing features of the film product so that the film product may be used efficaciously. For instance, use of the positioning feature to identify a feature of the film product may aid in positioning the film product to achieve its desired effect or function, such as by orienting the film product either independently of and prior to application to the treatment site, or during application to and with respect to the treatment site. Other functions may be performed or achieved by the use of a positioning feature in accordance with the principles of the present invention, the scope of the invention not being limited by the specific function of the film product or by the described exemplary orienting function of the positioning feature.

Turning to the drawings, exemplary film products 100, 200, 300, 400, 500, and 600 are illustrated in FIGS. 1-6 with exemplary respective positioning features 110, 210, 310, 410, 510, and 610. In the following description, elements or components similar to those in the embodiment of FIGS. 1 to 6 (though not necessarily having identical features) are designated with the same reference numbers increased by 100 for each increase in figure number and redundant description is omitted. It will be appreciated that the film product may be in any desired shape or form, and may be made of any desired material or composition. Thus, although the exemplary film products 100, 200, 300, and 400 illustrated in FIGS. 1-4 are rectangular, it will be appreciated that the principles of the present invention may be applied to a film product of any other desired shape, such as round, square, triangular, trapezoidal, irregular, etc. For instance, a film product may be formed with curvilinear sides, such as film product 500 of FIG. 5.

It will further be appreciated that the positioning feature to be provided on a film product in accordance with the principles of the present invention may be in any desired shape or form that achieves the desired purpose of distinguishing or differentiating features or aspects of the film product for proper use and/or application and/or functioning of the film product. For instance, the positioning feature preferably may be provided to identify a particular orientation of the film product itself, such as by identifying which surface is facing the user and which surface is facing the treatment site. The positioning feature may alternatively or additionally be provided simply for purposes of alignment at and/or with respect to the treatment site. The manner in which the positioning feature is provided, such as the form or location, need not be limited to the exemplary embodiments of FIGURES 1-6. Instead, it will be appreciated that the basic function of the positioning feature as distinguishing or differentiating features or aspects of the film product may be achieved without restricting the precise form of the positioning feature.

Turning to the exemplary embodiments of FIGS. 1-6, a positioning feature formed in accordance with the principles of the present invention may be formed in any of a variety of manners, including, without limitation, a mechanical/structural identifier, a visual indicator on a surface, or a textural feature on a surface. The film product may be provided with a positioning feature by being shaped or marked with a shape, cut-out, figure, color, hologram, mark, word, texture, or other indicia, which can uniquely identify sufficient information about the film product for the desired manipulation and/or orienting and/or general use of the film product. The type of positioning feature, as well as its location, may dictate the feature-distinguishing and indicating functions the positioning feature serves.

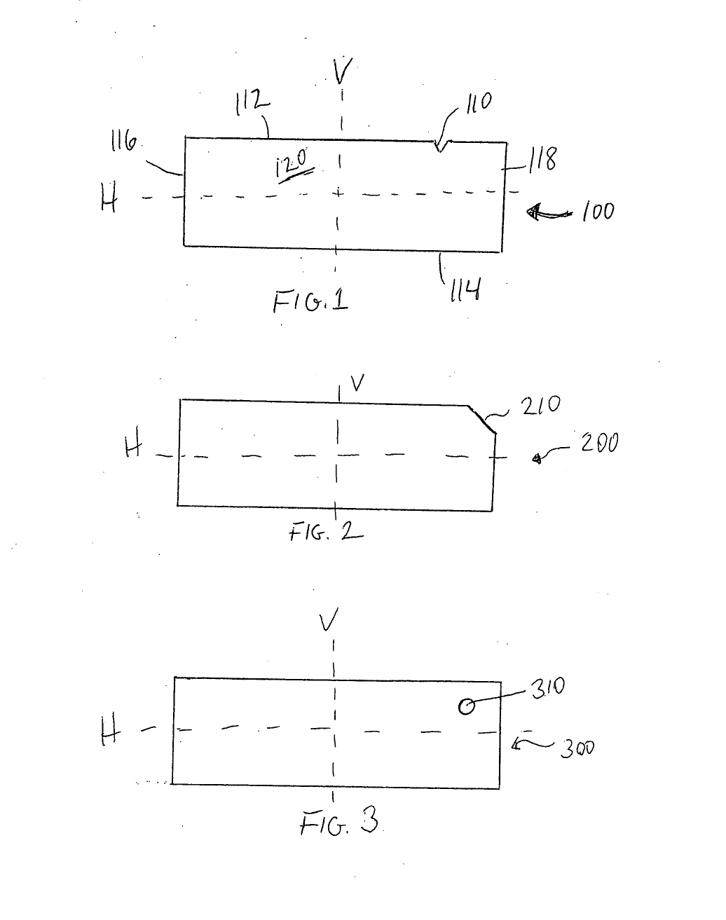

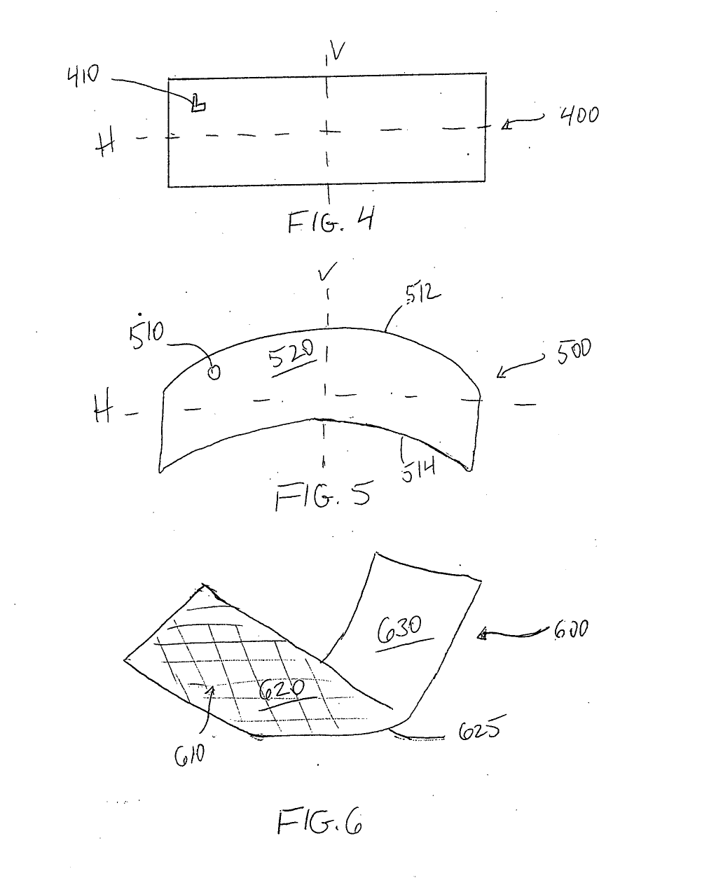

One simple embodiment of a positioning feature in accordance with the principles of the present invention is a mechanical or structural identifier (hereinafter, simply “mechanical identifier” for the sake of convenience and without intent to limit such term) such as positioning features 110, 210, 310, 410, 510 of FIGS. 1-5. Positioning features 110, 210, 310, 410, 510 are formed by mechanically altering the structure of the film product 100, 200, 300, 400, 500. For instance, an irregularity may be formed along an edge of the film product, as in FIGS. 1 and 2, or a cut-out may be formed through the film product spaced from the edges of the film product, as in FIGS. 3-5. More particularly, an irregularity along an edge of the film product may be formed in any desired shape, such as a notch 110 (FIG. 1) or a cut-off corner 210 (FIG. 2). Similarly, a cut-out may be formed in any desired shape, such as a round hole 310, 510 (FIGS. 3 and 5) or hole of another shape, such as an asymmetrical shape like L-shaped hole 410 (FIG. 4).

Positioning features in the form of a mechanical identifier by their very nature can indicate orientation of the film product with respect to a treatment site. However, a simple mechanical identifier cannot necessarily definitively indicate the orientation of the surfaces of the film product itself. This is because the presence alone of such an indicator as a mechanical identifier may not provide sufficient data to a user to be able to differentiate at least certain features of the film product. For example, if a film product is symmetrical about an axis of symmetry, then a mechanical identifier positioned along such axis of symmetry (especially a mechanical identifier that itself is symmetrical about such axis of symmetry as well) cannot necessarily serve to differentiate the opposing surfaces of the film product from each other. As another example, a round hole provided as a positioning feature in a round film product would not necessarily provide sufficient information to differentiate the opposing surfaces of the film product.

One manner of allowing a positioning feature in the form of a mechanical identifier to differentiate, more definitively, the surfaces of a film product is to locate the mechanical identifier at a readily identifiable position on the film product itself. Generally, the ability of a mechanical identifier to distinguish a surface of a film product is facilitated by basing selection of the location of the mechanical identifier on the shape of the film product. One useful principle is to place the mechanical identifier offset from any and all axes of symmetry. As discussed above, if the mechanical identifier is along an axis of symmetry of a film product it is difficult, if not impossible, to differentiate the opposite surfaces of the film product. Another useful principle is that the shape of the mechanical identifier can serve as a further indicator of the orientation of the film product on which the mechanical identifier is provided. Such principles may be better appreciated with reference to the exemplary embodiments of FIGS. 1-4, as will now be described.

Exemplary film products 100, 200, 300, and 400 have shapes that are symmetrical about a horizontal central axis and also about a vertical central axis.

Accordingly, it is desirable to locate a positioning feature 110, 210, 310, 410 in the form of a mechanical identifier offset from both the horizontal and vertical central axes so that the mechanical identifier may serve to differentiate the symmetrical sides of the film product.

Reference is made to film product 100 of FIG. 1 to illustrate this principle. Film product 100 has two pairs of opposite edges: opposite edges 112 and 114 (respectively top and bottom edges according to the orientation of film product 100 in FIG. 1), and opposite edges 116 and 118 (respectively left and right edges according to the orientation of film product 100 in FIG. 1). Positioning feature 110 is formed along top edge 112 at a location offset from central vertical axis V. In other words, positioning feature 110 is not located at a midpoint along the length of top edge 112. Moreover, because top edge 112 is, by its very nature as an edge, offset from central horizontal axis H, positioning feature 110, when provided along top edge 112, necessarily is also offset from central horizontal axis H as well. Accordingly, a user may be assured that when looking at film product 100 if positioning feature 110 is on the right side of top edge 112 of film product 100, then the surface 120 seen in FIG. 1 is the surface facing the user at that point. Rotation of film product 100 within the plane of the page does not alter such clear position indicating capability. For instance, if positioning feature 110 is located along what appears to be a bottom edge, on the left side thereof, it is also clear that surface 120 is facing the user. In contrast, if positioning feature 110 appears on the left side of top edge 112, then it is clear to the user that the surface opposite surface 120 is facing the user.

In contrast, exemplary film product 500 of FIG. 5 illustrates a film product that is symmetrical about only one axis of symmetry (vertical central axis V). The asymmetrical sides of film product 500 are inherently distinguishable from one another. Accordingly, positioning feature 510 need only be positioned offset from the axis of symmetry, axis V. So long as positioning feature 510 appears on the left side of film product 500 when convex edge 512 is a “top” edge and concave edge 514 is a “bottom” edge, it is clear that the surface 520 of film product 500 seen in FIG. 5 is the surface facing the user at that point as well.

Of course, complete symmetry, such as in a circular film product, complicates placement, as described above with respect to provision of a round hole as the positioning feature for a round film product. An asymmetrical positioning feature, such as the L-shaped positioning feature 410 of film product 400 in FIG. 4, may be used advantageously to permit orientation of the asymmetrical positioning feature to distinguish symmetrical sides from one another and thus to permit differentiation of the surfaces of a symmetrical film product.

As noted above, instead of providing a positioning feature in the form of a mechanical identifier, a positioning feature may be formed in accordance with the principles of the present invention as a surface feature which alters the surface of the film product. If the positioning feature is formed on a surface of the film product, then such positioning feature is helpful in differentiating opposing surfaces of the film product. In general, in contrast with a structural feature, a surface feature preferably does not extend through the material of the film product. Such a surface feature may be in any desired form that permits either visual or tactile differentiation of the surfaces of the film product. For instance, a visual indicator such as visible symbols, printed indicia, stippling, shading, or coloring may be used for ready visual differentiation of the surface bearing such visual indicator. Such visual indicator may be applied by any desired technique, including, without limitation, blotching, coloring, cutting, embossing, engraving, marking, printing (by ink jet, video jet, or flexographic printing, or any other desired technique known in the art), shaping, stamping. Additionally, or alternatively, surface texturing that alters the tactile features of the surface, such as by formation of non-smooth, unsmooth, or rough areas or regions (“textured” hereafter for the sake of convenience and without intent to limit), may be used to differentiate surfaces of the film product via tactile sensation.

Positioning features in the form of surface features are generally useful for film products that carry a particular active on only one surface thereof and only deliver the active at or via that surface. Such positioning feature may be particularly helpful in instances in which the surfaces are not readily distinguishable from each other. Examples of film products having surfaces that are not readily distinguishable include film products utilizing a dry adhesive such that a releasable backing strip is not needed to protect a sticky adhesive surface and thus cannot provide an indication of which surface bears the adhesive and is to be applied to the treatment site.

An exemplary embodiment utilizing a surface feature as a positioning feature is illustrated in FIG. 6. Film product 600 of FIG. 6 is illustrated folded over itself to show opposite surfaces 620 and 630. As may be appreciated, positioning feature 610 is a surface feature provided on only one of the surfaces of film product 600. As such, surface 620, bearing positioning feature 610, is readily distinguishable from surface 630.

Thus, it will be appreciated that the present invention facilitates use and/or application of a film product upon the user's location or identification of a positioning feature provided on the film product in accordance with the principles of the present invention. Once the positioning feature has been located or identified, the user can now differentiate or distinguish at least one feature of the film product. Such information may be employed by the user to use and/or manipulate and/or orient and/or apply the film product as desired. Film products incorporating positioning features in accordance with the principles of the present invention may be sold with instructions for the end users regarding locating the positioning feature, and identifying a desired feature of the film product based on information obtainable upon locating the positioning feature. The instructions may further provided guidance as to use of the film product in conjunction with location of the positioning feature.

As discussed earlier, it will be appreciated that the film products 100, 200, 300, 400, 500 may be any type of film product preferably configured to provide a therapeutic affect at a desired treatment site. One example of a film product that may embody the principles of the present invention is a medicated strip for delivering a systemic or topical active to a treatment site. A more specific example of such a medicated strip is one applied to teeth to whiten the teeth. The positioning features of the present invention are particularly helpful in assisting the user in orienting the medicated strip for proper application to his/her teeth. One exemplary tooth whitening strip utilizes a dry, moistenable adhesive and therefore can be applied directly to the user's teeth upon removal from a protective packaging or pouch without the need to remove a releasable backing strip or otherwise to alter the structure or material of the tooth whitening strip. Provision of a positioning feature on the tooth whitening strip facilitates orientation of the strip, which is essential if only one of the surfaces can deliver the whitening agent. It will, however, be appreciated that the principles of the present invention may be applied to film or strip products for use other than in oral cavities.

It will be appreciated that features described with respect to one embodiment typically may be applied to another embodiment, whether or not explicitly indicated. The various features hereinafter described may be used singly or in any combination thereof. Therefore, the present invention is not limited to only the embodiments specifically described herein. Moreover, the film compositions described in the examples disclosed in the parent applications as well as herein illustrate specific embodiments of the film compositions of the present invention, but are not intended to be limiting thereof. Other modifications can be undertaken by the skilled artisan without departing from the spirit and scope of the present invention

While the foregoing description and drawings represent exemplary embodiments of the present invention, it will be understood that various additions, modifications, and substitutions may be made therein without departing from the spirit and scope of the present invention. In particular, it will be clear to those skilled in the art that the present invention may be embodied in other specific forms, structures, arrangements, proportions, and with other elements, materials, and components, without departing from the spirit or essential characteristics thereof. One skilled in the art will appreciate that the principles of the present invention may be used with many modifications of structure, arrangement, proportions, materials, and components and otherwise, used in the practice of the invention, which are particularly adapted to specific environments and operative requirements without departing from the principles of the present invention. The presently disclosed embodiments are therefore to be considered in all respects as illustrative and not restrictive, the scope of the invention being indicated by the appended claims, and not limited to the foregoing description.

What is claimed is:

1. An article for delivering at least one active to a treatment site on a body, said article comprising:
a film product configured to deliver at least one active to the treatment site; and
a positioning feature provided on said film product to distinguish said film product orientation for positioning said film product at said treatment site in a selected orientation.

2. An article as in claim 1, wherein:
said film product has first and second opposing surfaces that are not readily distinguishable from each other; and
said positioning feature is provided to facilitate differentiating said first opposing surface from said second opposing surface of said film product.

3. An article as in claim 2, wherein said positioning feature comprises a surface feature on at least one of said first and second opposing surfaces.

4. An article as in claim 3, wherein said surface feature permits visual or tactile differentiation of said first and second opposing surfaces.

5. An article as in claim 4, wherein said surface feature is selected from the group consisting of: at least one visible symbol, printed indicia, stippling, shading, coloring, surface texturing, and combinations thereof.

6. An article as in claim 2, wherein said positioning feature comprises a mechanical identifier that structurally alters said film product to distinguish at least one feature of said film product.

7. An article as in claim 6, wherein said mechanical identifier is selected from the group comprising: an irregularity along an edge of said film product, and a hole or cutout formed through said film product.

8. An article as in claim 1, wherein said positioning feature is provided offset from any lines of symmetry of said film product.

9. An article as in claim 1, wherein said positioning feature is asymmetrical in shape.

10. An article for providing a therapeutic affect at a treatment site, said article comprising:
a film product configured for positioning on a body area to provide the therapeutic affect to said body area; and
a positioning feature provided on said film product to distinguish said film product orientation for positioning on said body area in a selected orientation.

11. An article as in claim 10, wherein said positioning feature comprises a surface feature on at least one of said first and second opposing surfaces.

12. An article as in claim 11, wherein said surface feature permits visual or tactile differentiation of said first and second opposing surfaces.

13. An article as in claim 10, wherein said positioning feature comprises a mechanical identifier that structurally alters said film product to distinguish at least one feature of said film product.

14. An article as in claim 10, wherein said positioning feature is provided offset from any lines of symmetry of said film product.

15. An article as in claim 10, wherein said positioning feature is asymmetrical in shape.

16. An article as in claim 10, wherein:
said film product has first and second opposing surfaces;
said film product comprises a film composition carrying at least one active on only one of said first and second opposing surfaces;
said first and second opposing surfaces are not readily distinguishable from each other; and
said positioning feature serves to distinguish said first and second opposing surfaces from each other.

17. A method of applying a film product to a treatment site, said method comprising:
locating a positioning feature on said film product; and
utilizing said positioning feature to orient said film product for application to said treatment site.

18. A method as in claim 17, further comprising orienting said film product to contact only one of two opposing surfaces of said film product to the treatment site.

19. A method as in claim 17, further comprising orienting said film product with respect to the treatment site.

20. A method as in claim 17, further comprising applying said film product to said treatment site without altering said film product.

21. A method as in claim 20, wherein altering said film product includes removing or adding a material from or to said film product.
